(19) United States
(12) Patent Application Publication
(10) Pub. No.: US 2026/0159888 A1
(43) Pub. Date: Jun. 11, 2026

(54) COMPOSITION FOR DIAGNOSING AGE-RELATED MACULAR DEGENERATION USING PROTEOMIC ANALYSIS AND BIOMARKERS, METHOD FOR PROVIDING INFORMATION FOR DIAGNOSING AGERELATED MACULAR DEGENERATION, AND METHOD FOR SCREENING SUBSTANCES FOR TREATING AGERELATED MACULAR DEGENERATION

(71) Applicant: JULIA LABORATORY INC., Suwon-si, Gyeonggi-do (KR)

(72) Inventor: Wan Jin JAHNG, Suwon-si, Gyeonggi-do (KR)

(73) Assignee: JULIA LABORATORY INC., Suwon-si, Gyeonggi-do (KR)

(21) Appl. No.: 18/723,459

(22) PCT Filed: Jul. 21, 2022
(86) PCT No.: PCT/KR2022/010711

§ 371 Date: Jun. 23, 2024

(30) Foreign Application Priority Data
Dec. 29, 2021 (KR) 10-2021-0190925

(51) Int. Cl. C12Q 1/6883; G01N 33/68

(52) U.S. Cl. CPC C12Q 1/6883; G01N 33/6893; C12Q 2600/136; C12Q 2600/158; G01N 2500/10; G01N 2800/16

(57) ABSTRACT

The present invention provides a composition for diagnosing age-related macular degeneration (AMD) using proteomic analysis and biomarkers, a method for providing information for diagnosing AMD, and a method for screening substances for treating AMD, wherein the composition for diagnosing age-related macular degeneration (AMD), includes a preparation for measuring the mRNA or protein expression level of an age-related macular degeneration-related biomarker.

### FIELD OF THE INVENTION

The present invention relates to a composition for diagnosing age-related macular degeneration (AMD) using proteomic analysis and biomarkers, a method for providing information for diagnosing AMD, and a method for screening substances for treating AMD.

### DISCUSSION OF RELATED ART

Age-related macular degeneration (AMD) is a progressive or multifactorial disease that causes the degeneration or atrophy of the retina and retinal pigment epithelium, or choriocapillaris in adults over the age of 50. AMD is a serious disease that has already surpassed diabetic retinopathy and glaucoma to become the leading cause of blindness in adults over the age of 50 in Western Europe. Eight million or more people in the United States are currently reported to have non-vascular or atrophic age-related macular degeneration (dry AMD), the more common form of the disease, and three million or more people lose their vision to the disease each year. Such a geriatric disease is likely to emerge not only as an economic problem in terms of medical expenses, but also as a major social problem in terms of deterioration in quality of life. Therefore, there is an urgent need for research into early diagnosis and efficient treatment of AMD.

However, the pathogenesis of AMD is multifactorial and only partially understood. Although a correlation with the accumulation of harmful environmental factors such as environmental pollution, excessive light exposure, and oxidative stress has been reported in addition to genetic predisposition, the specific mechanisms have not been yet fully understood.

There are two forms of AMD: non-exudative (dry) and exudative (wet). The non-exudative form, which accounts for the majority of AMD, refers to a case where lesions such as drusen or atrophy of the retinal pigment epithelium occur in the retina. The case does not usually induce severe vision loss, but may develop into a wet form. The exudative form refers to a case where choroidal neovascularization grows under the retina. This neovascularization may cause exudates, bleeding, and the like in the macula, affecting central vision, and may also lead to blindness within two months to three years after the occurrence. The exudative form of macular degeneration progresses very quickly, often causing rapid deterioration of vision within a few weeks. Generally, once visual disturbance begins with AMD, early detection is extremely important because it is often impossible to recover previous levels of vision.

Therefore, the present inventors have completed the present invention to elucidate the pathological mechanism of AMD by analyzing the correlation and interaction of various related proteins isolated from AMD ocular tissues such as the retinal pigment epithelium, and to provide a method for effectively diagnosing and treating AMD.

### DISCLOSURE

### Technical Goals

Therefore, a problem to be solved by the present invention is to provide a composition and method capable of effectively diagnosing age-related macular degeneration (AMD) by elucidating the mechanism of action of biomarkers through proteomic analysis related to AMD, and to provide a method for screening substances capable of treating AMD.

### Technical Solutions

To solve the above problem, the present invention provides a composition for diagnosing age-related macular degeneration (AMD) according to one embodiment.

The composition for diagnosing age-related macular degeneration includes a preparation for measuring the mRNA or protein expression level of an age-related macular degeneration-related biomarker, and the age-related macular degeneration-related biomarker includes one or more of the biomarkers as shown in the following Table 1.

TABLE 1

| | Major | Minor |
|---|---|---|
| Biomarker | category | category |
| Pyruvate kinase | 1 | 1-1 |
| α2 macroglobulin MG1 domain | 1 | 1-1 |
| PAK S/T kinase | 1 | 1-1 |
| Protein phosphatase (PP2A) | 1 | 1-1 |
| Tyrosine protein kinase (FES/FPS) | 1 | 1-1 |
| Creatine kinase | 1 | 1-1 |
| Ubiquinone oxidoreductase | 1 | 1-2 |
| Inositol 1,4,5-trisphosphate receptor | 1 | 1-2 |
| Calponin | 1 | 1-2 |
| Ankyrin repeat domain 30B | 1 | 1-2 |
| Guanylate cyclase | 1 | 1-2 |
| Complement factor family members: complement | 1 | 1-2 |
| component 3 (C3), complement component 9 | | |
| (C9), complement factor H (CFH), and | | |
| vitronectin | | |
| Retinoid-binding proteins: retinal pigment | 1 | 1-2 |
| epithelium-specific 65 (RPE65), retinal G | | |
| protein coupled receptor (RGR), and | | |
| retinaldehyde binding protein (RLBP) | | |
| Mitochondrial protein involved in energy | 1 | 1-2 |
| metabolism (ATPase) | | |
| Transcription factor (p53) | 1 | 1-2 |
| Protease (MMP) | 1 | 1-2 |
| Cytoskeletal polymers: lamin, albumin | 1 | 1-2 |
| Apoptotic protein (Annexin V) | 1 | 1-2 |
| Redox reaction (GST) protein | 1 | 1-2 |
| Ubiquitin | 1 | 1-2 |
| Peroxiredoxin | 1 | 1-2 |
| MAP kinase | 1 | 1-2 |
| Bub1/Bub3 complex | 1 | 1-2 |
| Nuclear signaling factor (p53) | 1 | 1-3 |
| Nuclear signaling factor (BRCA1) | 1 | 1-3 |
| Nuclear signaling factor (ATM) | 1 | 1-3 |
| Oxygen-mediated apoptotic molecule (HIF) | 1 | 1-3 |
| Nuclear-mitochondrial signaling factor | 1 | 1-3 |
| (VDAC) | | |
| Nuclear-mitochondrial signaling factor | 1 | 1-3 |
| (EP300) | | |
| Vimentin | 1 | 1-3 |
| Transferrin | 2 | 2-1 |
| Enolase | 2 | 2-1 |
| Glial fibrillary acidic protein (GFAP) | 2 | 2-1 |
| Dynamin-like protein | 2 | 2-1 |
| WD protein 59 | 2 | 2-1 |
| Apoptotic molecules: Bcl2, BAD, BAX, BAK | 2 | 2-2 |
| Cyclooxygenase (COX) | 2 | 2-2 |
| Nuclear signaling factor (SIRT1) | 2 | 2-3 |
| Aging-related protein (prohibitin) | 2 | 2-3 |
| Oxygen-mediated apoptotic molecule (EPO) | 2 | 2-3 |
| Oxygen-mediated apoptotic molecule (Bcl) | 2 | 2-3 |
| Oxygen-mediated apoptotic molecule (BAD) | 2 | 2-3 |
| Oxygen-mediated apoptotic molecule (TXN) | 2 | 2-3 |
| Nuclear-mitochondrial signaling factor | 2 | 2-3 |
| (PHB) | | |
| Nuclear-mitochondrial signaling factor | 2 | 2-3 |
| (RBP) | | |

A preparation for measuring the mRNA expression levels of the biomarkers as shown in Table 1 may include one or more of a primer pair, a probe, and an antisense nucleotide, which specifically bind to the gene of the biomarker. Since the nucleic acid information on the biomarker genes is known from GeneBank and the like, a person skilled in the art may design their primer pairs, probes or antisense nucleotides based on the sequences. The probe refers to a substance capable of confirming the presence of a target substance to be detected in a sample by specifically binding to the target substance, and may be a peptide nucleic acid (PNA), a locked nucleic acid (LNA), a peptide, a polypeptide, a protein, RNA, or DNA, but is not limited thereto.

Examples of a method for measuring the mRNA expression level include polymerase chain reaction (RT-PCR), competitive RT-PCR, real time RT-PCR, RNase protection assay (RPA), Northern blotting, DNA chips, and the like, but are not limited thereto.

Further, a preparation for measuring the protein expression level of the biomarker may include one or more of an antibody, an interacting protein, a ligand, nanoparticles, and an aptamer, which specifically bind to the protein or peptide fragment of the biomarker.

The antibody may include all of a polyclonal antibody, a monoclonal antibody, and a recombinant antibody.

To solve another problem, the present invention provides a kit for diagnosing age-related macular degeneration according to one embodiment. The kit for diagnosing age-related macular degeneration includes the composition for diagnosing age-related macular degeneration as described above. The kit for diagnosing age-related macular degeneration may be a reverse transcription polymerase chain reaction (RT-PCR) kit, a DNA chip kit, an enzyme linked immunosorbent assay (ELISA) kit, a protein chip kit, a rapid kit, or a multiple reaction monitoring (MRM) kit.

In addition, the kit may be used for mass spectrometry. In this case, the specific amino acid residue of the protein may have a modification such as myristoylation, isoprenylation, prenylation, glypiation, lipoylation, acylation, alkylation, methylation, demethylation, amidation, ubiquitination, phosphorylation, deamidation, glycosylation, oxidation, or acetylation.

The kit for diagnosing age-related macular degeneration may be prepared by a method known to the person skilled in the art. The kit for diagnosing age-related macular degeneration may include, for example, an antibody in lyophilized form and a buffer, a stabilizer, an inert protein, and the like. The antibodies may be labeled with radionuclides, fluorescors, enzymes, and the like.

To solve still another problem, the present invention provides a method for providing information for diagnosing age-related macular degeneration according to one embodiment. The method for providing information for diagnosing age-related macular degeneration includes: (a) measuring the mRNA or protein expression level of an age-related macular degeneration-related biomarker in a biological sample of a subject; (b) comparing the measured mRNA or protein expression level with that of a control sample; and (c) diagnosing the subject as being likely to have age-related macular degeneration when the measured mRNA or protein expression level is increased or decreased compared to that of the control sample. The age-related macular degeneration-related biomarker is one or more of the biomarkers in Table 1 above.

Specifically, biomarkers belonging to the first biomarker in Table 1 above are characterized in that their mRNA or protein expression levels are increased in age-related macular degeneration cells or patients compared to a normal control. On the other hand, biomarkers belonging to the second biomarker are characterized in that their mRNA or protein expression levels are decreased in age-related macular degeneration cells or patients compared to the normal control. Therefore, when the mRNA or protein expression level of the first biomarker is increased compared to the control sample or the mRNA or protein expression level of the second biomarker is decreased compared to the control sample, the subject may be diagnosed as being likely to have age-related macular degeneration. That is, it is possible to diagnose (or determine, judge) that the subject is highly likely to have age-related macular degeneration or to develop it.

Each biomarker in Table 1 above may be derived from a different site. Biomarker 1-1 and biomarker 2-1 may be derived from the neuroretina of a subject, and biomarker 1-2 and biomarker 2-2 may be derived from the retinal pigment epithelium (RPE) of the subject. Therefore, a more accurate diagnosis can be achieved by measuring and comparing the mRNA or protein expression levels of biomarkers 1-1 and/or 2-1 when a biological sample is derived from the neuroretina of a subject, and by measuring and comparing the mRNA or protein expression levels of biomarkers 1-2 and/or 2-2 when the biological sample is derived from the retinal pigment epithelium (RPE) of the subject.

Meanwhile, biomarkers 1-3 and 2-3 may be derived from the neuroretina and/or the retinal pigment epithelium.

Furthermore, at least some of the biomarkers may be those in which some amino acids are phosphorylated. Specifically, the age-related macular degeneration-related biomarker may include one or more selected from the group consisting of a transferrin in which S678 and/or S409 are/is phosphorylated; a pyruvate kinase in which S437 is phosphorylated; WD protein 59 in which S375 and/or Y437 are/is phosphorylated; and ankyrin repeat domain 30B in which S592 is phosphorylated.

The mRNA or protein expression levels of the biomarkers may be measured and compared using the above-described composition or kit for diagnosing age-related macular degeneration.

Further, steps (a) to (c) may be analyzed using a statistical method or algorithm in order to improve the accuracy of diagnosis, and it is possible to use one or more analysis methods selected from the group consisting of a linear or non-linear regression analysis method, a linear or non-linear classification analysis method, a logistic regression analysis method, analysis of variance (ANOVA), a neural network analysis method, a genetic analysis method, a support vector machine analysis method, a hierarchical analysis or clustering analysis method, a hierarchical algorithm or Kernel principal component analysis method using a decision tree, a Markov Blanket analysis method, a recursive feature elimination or entropy-based recursive feature elimination analysis method, and a forward floating search or backward floating search analysis method.

To solve the above problems, the present invention provides a method for treating age-related macular degeneration, further including (d) administering a therapeutic agent capable of reducing a biomarker whose mRNA or protein expression level is measured in step (a) according to another exemplary embodiment. That is, when it is determined that a subject has a possibility of having age-related macular degeneration because a specific biomarker is increased or decreased through steps (a) to (c), the subject may be treated by administering a therapeutic agent capable of directly or indirectly inhibiting or promoting the corresponding biomarker.

The therapeutic agent may be administered in the form of a pharmaceutical composition, and may be prepared in a dosage form such as an injection by mixing with a pharmaceutically acceptable carrier, excipient, diluent, and the like according to methods publicly known in the pharmaceutical field and administered by intravenous injection, and the like.

The method for treating age-related macular degeneration of the present invention may further include (e) measuring again the mRNA or protein expression level of the biomarker after administration of the pharmaceutical preparation in step (d).

To solve yet another problem, the present invention provides a method for screening substances for treating age-related macular degeneration according to one embodiment. The method for screening substances for treating age-related macular degeneration includes: (a) treating neuroretinal cells or retinal pigment epithelial cells with a candidate substance; (b) measuring the mRNA or protein expression levels of one or more of the biomarkers in Table 1 above in the treated cells; and (c) comparing the measured mRNA or protein expression levels with those of control cells to confirm whether the first biomarker is decreased and/or whether the second biomarker is increased.

Since the first and second biomarkers are substances which are increased and decreased, respectively, compared to a normal control, when the first or second biomarker is treated with a specific candidate substance and decreased or increased, respectively, the corresponding candidate substance is determined to be a substance which is likely to treat age-related macular degeneration, and may be used to carry out a next-stage experiment, such as preclinical and clinical trials.

The treating of the neuroretinal cells or retinal pigment epithelial cells with the candidate substance may be performed by one or more methods of in vitro, in vivo, ex vitro, ex vivo, and in silico, and the neuroretinal cells or retinal pigment epithelium may be isolated or derived from an animal model or a human.

Specific details of other embodiments are included in the detailed description.

### Advantageous Effects

According to embodiments of the present invention, by using specific biomarkers related with age-related macular degeneration (AMD), it is possible to effectively diagnose AMD and to screen for a substance for treating AMD.

The effects according to the embodiments of the present invention are not limited to the contents exemplified above, and more various effects are included in the present specification.

### BRIEF DESCRIPTION OF DRAWINGS

FIGS. 1 to 4 are graphs showing the 3D analysis results of AMD proteins in the neuroretina and RPE according to an experimental example of the present invention; and

FIG. 5 is a protein map prepared based on the analysis of AMD-related proteins according to an experimental example of the present invention.

### BEST MODE FOR CARRYING OUT THE INVENTION

The terms used in the present specification are used merely to describe embodiments, and are not intended to limit the present invention. In the present specification, the ‘and/or’ includes each and all combinations of one or more of the items mentioned. Further, the singular form also includes the plural forms unless specifically stated in a phrase. The terms ‘comprises’ and/or ‘comprising’ used in the specification do not exclude the presence or addition of one or more other constituent elements in addition to the referenced constituent elements.

Further, in describing the constituent elements of the examples of the present invention, terms such as first, second, A, B, (a), and (b) may be used. These terms are merely for distinguishing one constituent element from another, and the nature, turn, or order of the corresponding constituent element is not limited by the term.

As used herein, ‘prevention’ refers to suppressing or delaying the onset of a symptom or disease in an individual who does not yet have the symptom or disease, but may suffer from the symptom or disease.

As used herein, ‘treatment’ refers to all actions that ameliorate or beneficially change a symptom from an individual, and refers to, for example, (a) suppression of the development (exacerbation) of a symptom or disease, (b) reduction or amelioration of a symptom or disease, or (c) elimination of a symptom or disease.

As used herein, ‘individual’ or ‘subject’ refers to a cell, tissue, animal, or human in which a particular disease or disorder is to be treated using the present invention.

As used herein, ‘diagnosis’ refers to any action capable of providing information related to a particular disease or disorder, such as the action of determining the presence or absence of the disease or disorder or the risk of developing the disease or disorder, as well as confirming the characteristics of a pathological state, using the composition or kit of the present invention.

As used herein, ‘being likely to have a disease’ refers to inclusion of both being likely to have developed a particular disease or disorder and being likely to develop the disease or disorder

As used herein, ‘biological sample’ refers to blood, plasma, serum, cells, tissue, body fluid, saliva, urine, aqueous humor, anterior chamber fluid, and the like of a subject.

As used herein, ‘derived from’ may not only refer to being isolated from a specific subject or a specific site, but also refer to the subject or site itself.

As used herein, ‘biomarker’ includes an organic biomolecule such as a peptide, a polypeptide, a nucleic acid, a lipid, a glycolipid, a glycoprotein, and a sugar (a monosaccharide, a disaccharide, an oligosaccharide, and the like), which show a significant increase or decrease pattern in gene or protein expression levels in an individual with a specific disease or disorder compared to a normal control (an individual without a specific disease or disorder).

As used herein, ‘control’ refers to a normal cell or patient who is free of or has not been induced to have a specific disease or disorder.

Hereinafter, embodiments of the present invention will be described in detail with reference to Preparation Examples and Experimental Examples, but it is obvious that the effects of the present invention are not limited by the following Experimental Examples.

### Experimental Example 1: Isolation and Analysis of AMD-Related Proteins from Animal Models

Sprague-Dawley rats (male, 250 to 300 g) and mice (C57/BL6J) were purchased from Charles River Laboratories Inc. (Wilmington, MA). As a positive control for retinal oxidative stress experiments, a diabetic state was induced by intravenous injection of streptozotocin (65 mg/kg in 0.1 M sodium citrate, pH 4.5, Sigma-Aldrich, St. Louis, MO). Negative control animals were administered a single injection of vehicle. The rats and mice (n=9, biological and technical triplicate experiments) were considered diabetic at blood glucose levels >350 mg/dL. Two or four weeks after the onset of diabetes, the animals were euthanized by anesthesia overdose. The control rats and mice were analyzed at 12 weeks of age, and aged rats and mice were analyzed at 52 and 54 weeks. Retinas from rats and mice were removed and frozen in liquid nitrogen. Retinas from the other groups were collected and prepared for biochemical analysis.

Stat View software was used for statistical analysis. Statistical significance was analyzed using unpaired Student's t-test or analysis of variance (ANOVA) when appropriate. A significance level of P<0.05 was considered statistically significant.

Further, to identify oxidative stress biomarkers, changes in specific cytoskeletal proteins in vivo were confirmed using an animal model (C3H female mice, 7 weeks). It was confirmed whether neurofilament, vimentin, and β-tubulin were overexpressed under 24-hour constant light conditions compared to 12-hour dark/12-hour light conditions.

### Experimental Example 2: Isolation and Analysis of AMD-Related Proteins from Human Donated Eyes

Tissues from eye donors were used in accordance with the tenets of the Declaration of Helsinki. Retinal tissues from humans with diabetic retinopathy (DR) (n=9, biological and technical triplicate experiments) were provided by the Georgia Eye Bank (Atlanta, GA). Age-related macular degeneration (AMD) retina (8 mm macular and peripheral punches), retinal pigment epithelial (RPE) cells (8 mm central and peripheral punches), and age-matched control eyes (n=9, biological and technical triplicate experiments) were provided by the Lions Eye Bank (Moran Eye Center, University of Utah). Proteomes from the macula (I), peripheral retina (II), central epithelial cells (III), and peripheral epithelial cells (IV) were compared with age-matched control donor eyes to determine aging-related molecular mechanisms in each region during the progression of macular degeneration. Retinal proteins were enriched by charge-based spin column chromatography and resolved by 2D gel electrophoresis. Trypsin-digested peptides from whole lysates were enriched using Ga³⁺/TiO₂ immobilized metal ion chromatography. Eluted proteins were analyzed using liquid chromatography. Serine, threonine, and tyrosine phosphorylation was confirmed by phospho-Western blot analysis.

### Experimental Example 3: Isolation and Analysis of AMD-Related Proteins from In Vitro Cells

For in vitro experiments of ARPE-19 and HRP, retinal pigment epithelial cells (ARPE-19) were purchased from ATCC (Manassas, VA) and human retinal progenitor (HRP) cells were kindly donated by Dr. Harold J. Sheeldo at the University of North Texas Health Science Center. ARPE-19 and HRP cells were cultured in a 5% CO₂ incubator at 37° C. in 100-mm dishes (Nalge Nunc International, Naperville, IL) using Dulbecco's modified Eagle's medium (DMEM) containing fetal bovine serum (10%) and penicillin/streptomycin (1%). Confluent cells were trypsinized (5 to 7 minutes at 37° C.) using a trypsin-EDTA buffer (0.1%, Sigma-Aldrich, St. Louis, MO), followed by centrifugation (300×g, 7 minutes). Cells (eight to nine passages) were grown to confluence for 2 to 4 days and then were treated with H₂O₂ (200 μM), intense light (7000 to 10,000 lux, 1 to 24 hours) or constant light (700 lux, 48 hours). HRP and ARPE-19 cells were washed with Modified Dulbecco's PBS and lysed using an IP lysis buffer containing Tris (25 mM), NaCl (150 mM), EDTA (1 mM), NP-40 (1%), protease inhibitor cocktail, and glycerol (5%). Then, by culturing the cells on ice for 5 minutes with periodic sonication (3×5 minutes), followed by centrifugation (13,000×g, 10 minutes), proteins (1 mg/ml, 200 to 400 μL) were loaded for immunoprecipitation at pH 7.4 and non-specific bindings were avoided using a control agarose resin cross-linked by 4% bead agarose. Amino group-linked protein-A beads were used to immobilize an antiprohibitin antibody with a coupling buffer (1 mM sodium phosphate, 150 mM NaCl, pH 7.2), followed by incubation (room temperature, 2 hours) with sodium cyanoborohydride (3 μL, 5 M). Columns were washed using a washing buffer (1 M NaCl), and a protein lysate was incubated in the protein A-antibody column with gentle rocking overnight at 4° C. Unbound proteins were isolated by flowing a washing liquid, and the columns were washed three times using a washing buffer to remove non-specific binding proteins. Interacting proteins were eluted by incubating with an elution buffer at room temperature for 5 minutes. The eluted proteins were reduced by a Laemmli sample buffer (5λ, 5% β-mercaptoethanol). The eluted proteins were isolated using SDS-PAGE and stained using Coomassie blue (Pierce, IL) or a silver staining kit (Bio-Rad, Hercules, CA). Bound proteins were used to establish an interactome in this experiment.

Protein samples were purified with a ReadyPrep 2-D Cleanup Kit (Bio-Rad, CA) and quantified using a BCA protein assay kit (Pierce, Rockford, IL). 150 g of protein was incubated in 200 μl of a rehydration buffer (8 M urea, 2 M thiourea, 2% CHAPS and 50 mM DTT) supplemented with a 0.5% destreak IPG buffer, pH 3 to 10 (GE Healthcare, PA). Isoelectric focusing was performed using 11 cm immobilized dry strips with linear pH 3 to 10 and 4 to 7 gradients (Bio-Rad, CA). The strips were rehydrated at 20° C. for 12 hours. Proteins were isolated by Ettan IPGphor-3 (GE Healthcare) using a programmed voltage gradient at 20° C. for a total of 12 kVh (1 hour at 500 V, 1 hour at 1000 V, 2 hours at 6000 V and 40 minutes at 6000 V). The IPG strips were reduced with equilibration buffer I (0.375 M Tris-HCl, pH 8.8, 6 M urea, 20% glycerol, 2% SDS and 50 mM DTT) at 25° C. for 20 minutes and alkylated with an equilibration buffer for 20 minutes. Strips containing 150 mM iodoacetamide were placed on top of a polyacrylamide gel (8 to 16%, Bio-Rad, CA) and sealed with a 1% agarose buffer. Proteins were analyzed by Coomassie staining using Imperial Protein Stain (Pierce, Rockford, IL) and Western blot.

Protein bands were excised (1×1×1 mm) and then cultured using a Coomassie destaining buffer (200 μL of 50% MeCN in 25 mM NH₄HCO₃, pH 8.0, room temperature, 20 minutes) or a silver destaining buffer (50% of 30 mM potassium ferricyanide). Gel pieces were dehydrated (200 μL, MeCN) and vacuum-dried (Speed Vac, Savant, Holbrook, NY). Cysteine sulfide bonds in the protein were reduced with DTT (10 mM) in NH₄HCO₃ buffer (100 mM) at 56° C. for 30 minutes, and then alkylated with iodoacetamide (55 mM) in NH₄HCO₃ buffer (100 mM) at room temperature for 20 minutes. Proteins were digested using trypsin (13 ng/μL sequencing grade from Promega, 37° C., 10 hours) in NH₄HCO₃ (10 mM) containing MeCN (10%). The peptides were enriched using a buffer (50 μL of 50% MeCN in NH₄HCO₃, 5% formic acid, 20 minutes, 37° C.). Dried peptides were dissolved in a mass spectrometry sample buffer (5 to 10 μL, 75% MeCN in NH₄HCO₃, 1% trifluoroacetic acid). Alpha-cyano-4-hydroxycinnamic acid (5 mg/mL, MW 189.04, Sigma-Aldrich, St. Louis, MO) was freshly dissolved in a matrix buffer (50% MeCN, 50% NH₄HCO₃, 1% trifluoroacetic acid) and centrifuged (13,000×g, 5 minutes). A peptide-matrix mixture (0.5 μL) was spotted onto a MALDI plate (Ground steel, Bruker Daltonics, Germany). Mass spectrometer and all spectra were calibrated using a known peptide trypsin (842.5099 Da, 2211.105 Da). The mass spectrum was recorded in a range of 800 to 3000 Da using a Flex MALDI-TOF mass spectrometer (Bruker Daltonics, Germany, 70 to 75% laser intensity, 100 to 300 shots). Mass spectrometry data was analyzed using Flex analysis software (Bruker Daltonics, Germany). Peptides were analyzed using the Mascot software (Matrix Science) and NCBI/SwissProt database (zero mismatch cleavage, carbamidomethyl cysteine, methionine oxidation, 50 to 300 ppm mass tolerance, S/T/Y phosphorylation=79 m/z search). Peptide identification was evaluated based on the Mascot MOWSE score, the number of matched peptides, and the protein sequence coverage. MOWSE score is expressed as −10 log P as a probability value to compute the composite probability P.

AMD proteins were reconfirmed by the following experiment. To determine phosphorylation signals in retinal cells, ARPE19 cells were treated with 1) 200 μM H₂O₂, 2) intense light, or 3) light exposed for a long period of time. Protein changes were analyzed under oxidative stress conditions of intense light (7,000 to 10,000 lux) or long-term light (48 hours). Proteins were extracted, isolated by two-dimensional electrophoresis, and stained with Coomassie blue. Proteins from PBS-treated RPE cells or RPE cells grown under normal light conditions were used as controls. Proteins were analyzed using a MALDI-TOF and MALDI-TOF-TOF mass spectrometer.

### Experimental Example 4: Analysis Results of AMD-Related Proteins and Elucidation of Biomarkers

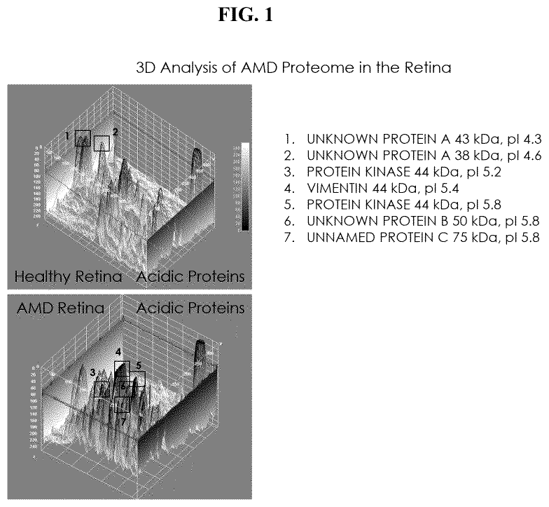

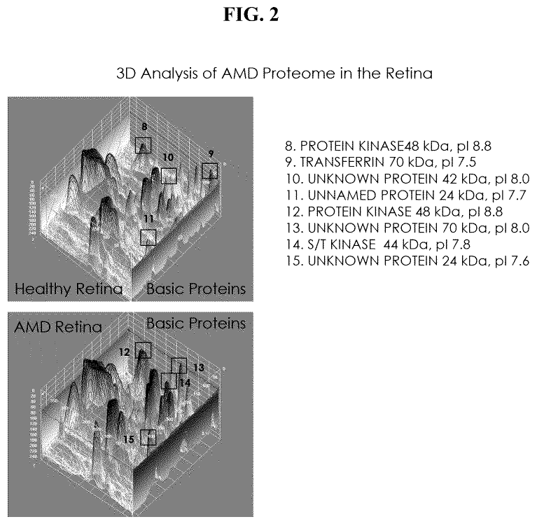

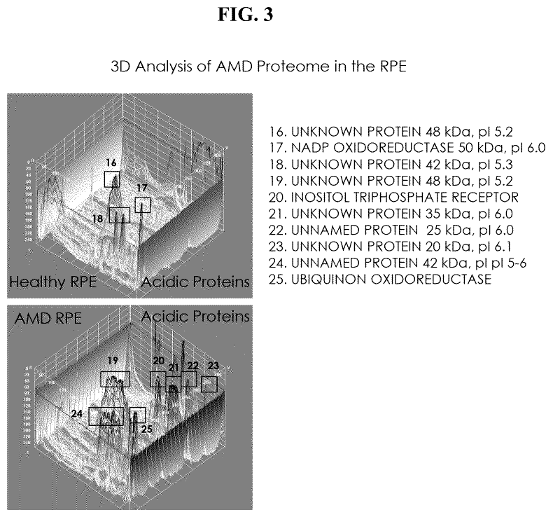

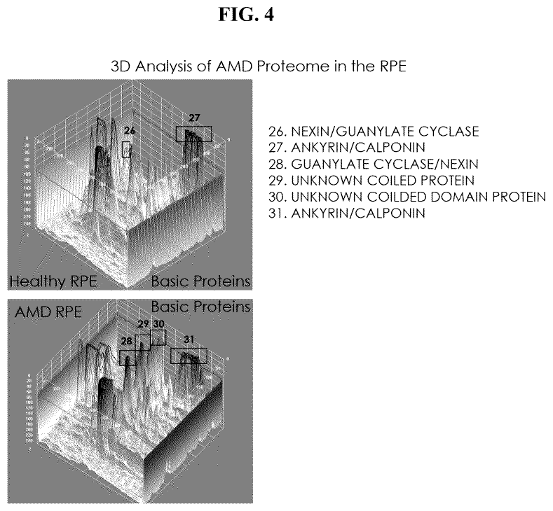

FIGS. 1 to 4 are graphs showing the 3D analysis results of AMD proteins in the retina and RPE.

First, 11 types of proteins were confirmed to be changed in the peripheral region of the AMD retina (neuroretina), as shown in the following Table 2.

TABLE 2

| | | Isolated | |
|---|---|---|---|
| No. | Protein | region | Change |
| 1 | Transferrin | Peripheral | Decreased |
| 2 | Pyruvate kinase | region of | Increased |
| 3 | Enolase | AMD retina | Decreased |
| 4 | Unnamed protein JL1 (α2 | (neuroretina) | Increased |
| | macroglobulin MG1 domain) | | |
| 5 | PAK S/T kinase | | Increased |
| 6 | Protein phosphatase (PP2A) | | Increased |
| 7 | Glial fibrillary acidic protein (GFAP) | | Decreased |
| 8 | Tyrosine protein kinase (FES/FPS) | | Increased |
| 9 | Dynamin-like protein | | Decreased |
| 10 | Creatine kinase | | Increased |
| 11 | Vimentin | | Increased |

The changes in the expression levels of the proteins as described above mean that in the AMD mechanism, iron homeostasis, energy regeneration, glycolysis, and cytoskeletal structure may be affected during AMD progression. In contrast, denatured proteins from peripheral RPE were identified, including the six proteins and the like shown in the following Table 3.

TABLE 3

| | | Isolated | |
|---|---|---|---|
| No. | Protein | region | Change |
| 12 | Ubiquinone oxidoreductase | Peripheral | Increased |
| 13 | Inositol 1,4,5-trisphosphate receptor | region of | Increased |
| 14 | Calponin | AMD RPE | Increased |
| 15 | Ankyrin repeat domain 30B | | Increased |
| 16 | Guanylate cyclase | | Increased |
| 17 | Unnamed protein JL2 | | Increased |

The changes in protein biomarkers as described above indicate mechanisms such as energy conversion, oxidative damage, blood flow, physiological hypoxia, cGMP and alterations in cell polarity during AMD progression. In addition, specific peptides were analyzed using a concentration protocol through gallium ion (Ga³⁺) chromatography to identify proteins as shown in the following Table 4. The following Table 4 includes information on the phosphorylation positions of proteins.

TABLE 4

| | | Isolated | |
|---|---|---|---|
| No. | Protein | region | Change |
| 18 | Transferrin (S678, S409) | Peripheral | Decreased |
| 19 | Unknown protein JL 3 (S140) | region of | Increased |
| 20 | Pyruvate kinase (S437) | AMD retina | Increased |
| 21 | WD protein 59 (S375, Y437) | (neuroretina) | Decreased |
| 22 | Ankyrin repeat domain 30B (S592) | Peripheral | Increased |
| 23 | Unknown protein JL4 (S770) | region of | Increased |
| | | AMD RPE | |

The proteins in the following Table 5 were identified as AIVD target proteins.

TABLE 5

| | | Isolated | |
|---|---|---|---|
| No. | Protein | region | Change |
| 24 | Complement factor family members | Peripheral | Increased |
| | (C3, C9, CFH, vitronectin) | region of | |
| | | AMD RPE | |
| 25 | Retinoid-binding proteins (RPE65, | Peripheral | Increased |
| | RGR, RLBP) | region of | |
| | | AMD RPE | |
| 26 | Apoptotic molecules (Bcl2, BAD, | Peripheral | Decreased |
| | BAX, BAK) | region of | |
| | | AMD RPE | |
| 27 | Mitochondrial protein involved in | Peripheral | Increased |
| | energy metabolism (ATPase) | region of | |
| | | AMD RPE | |
| 28 | Transcription factor (p53) | Peripheral | Increased |
| | | region of | |
| | | AMD RPE | |
| 29 | Protease (MMP) | Peripheral | Increased |
| | | region of | |
| | | AMD RPE | |
| 30 | Cytoskeletal polymers (lamin, | Peripheral | Increased |
| | albumin) | region of | |
| | | AMD RPE | |

Through the image analysis of the reconfirmation experiment, it was confirmed that 17 proteins were overexpressed and 7 proteins were decreased in an oxidative stress environment. Proteins were analyzed using a MALDI-TOF and MALDI-TOF-TOF mass spectrometer. Among these, retinoid metabolism-related proteins, including RPE65, an all-trans-retinylester isomerohydrolase, and retinol-binding protein, exhibited perturbed retinoid metabolism under oxidative stress in the RPE. As target proteins of AMD, an apoptotic protein (Annexin V) and a redox reaction (GST) protein were overexpressed, and aging-related (prohibitin) and cyclooxygenase (COX) proteins were decreased in RPE cells, as shown in the following Table 6.

TABLE 6

| No. | Protein | Isolated region | Change |
|---|---|---|---|
| 31 | Apoptotic protein (Annexin V) | ARPE19AMD RPE | Increased |
| | | peripheral region | |
| 32 | Redox reaction (GST) protein | ARPE19AMD RPE | Increased |
| | | peripheral region | |
| 33 | Aging-related protein | ARPE19Mouse | Decreased |
| | (prohibitin) | retinaAMD RPE | |
| | | peripheral region | |
| 34 | Cyclooxygenase (COX) | ARPE19AMD RPE | Decreased |
| | | peripheral region | |

Potential biomarkers under oxidative stress were analyzed by an interaction network. Overexpressed or underexpressed proteins under various stresses were calculated by STRING software for quantitative analysis of chemical binding. The oxidative stress interactome showed chemical bonds between factors as shown in the following Table 7.

TABLE 7

| | | Isolated | |
|---|---|---|---|
| No. | Protein | region | Change |
| 35 | Nuclear signaling factor (p53) | ARPE19Mouse | Increased |
| 36 | Nuclear signaling factor (BRCA1) | retinaAMD | Increased |
| 37 | Nuclear signaling factor (ATM) | retina | Increased |
| 38 | Nuclear signaling factor (SIRT1) | (neuroretina) | Decreased |
| 39 | Oxygen-mediated apoptotic molecule | peripheral | Decreased |
| | (EPO) | region | |
| 40 | Oxygen-mediated apoptotic molecule | AMD RPE | Increased |
| | HIF | peripheral | |
| 41 | Oxygen-mediated apoptotic molecule | region | Decreased |
| | (Bcl) | | |
| 42 | Oxygen-mediated apoptotic molecule | | Decreased |
| | (BAD) | | |
| 43 | Oxygen-mediated apoptotic molecule | | Decreased |
| | (TXN) | | |
| 44 | Nuclear-mitochondrial signaling | | Decreased |
| | factor PHB | | |
| 45 | Nuclear-mitochondrial signaling | | Increased |
| | factor (VDAC) | | |
| 46 | Nuclear-mitochondrial signaling | | Increased |
| | factor (EP300) | | |
| 47 | Nuclear-mitochondrial signaling | | Decreased |
| | factor (RBP) | | |

In addition, through oxidative stress biomarkers and AMD target proteins, it was confirmed that the factors as shown in the following Table 8 are involved in the progression of AMD. This indicates that the cytoskeletal protein phosphorylation, protein aggregation and mitochondrial signaling contribute to RPE cell death. In particular, neurofilament, vimentin, and β-tubulin were overexpressed under 24-hour constant light conditions compared to the 12-hour dark/12-hour light conditions. The lipid composition of RPE cells determined lipid changes under oxidative stress. Mass spectrometry revealed that the longer chain fatty acids located at sn-2 of phosphatidylcholine increased from 16 carbons to 24/26 carbons, and cholesteryl esters were also subjected to oxidative stress. The fatty acid saturation degree of phosphatidylcholine was also increased.

TABLE 8

| | | Isolated | |
|---|---|---|---|
| No. | Protein | region | Change |
| 48 | Ubiquitin | ARPE19 | Increased |
| 49 | Peroxiredoxin | ARPE19 | Increased |
| 50 | MAP kinase | ARPE19 | Increased |
| 51 | BUB 1/3 | ARPE19 | Increased |
| 52 | Vimentin | ARPE19 | Increased |

Taking into account the results of analyzing these AMD and oxidative stress interactomes, the mechanism of AMD may be defined as follows. AMD energy interactomes exhibit altered phosphocreatine shuttle, suggesting altered ATP-requiring reactions and the maintenance of a high ATP/ADP ratio in AMD.

Rhodopsin phosphorylation indicates increased Na⁺/K⁺ ATPase, phosphatidylinositol/inositol triphosphate signaling, and cGMP metabolism in AMD. Creatine kinase pumps out Na⁺ at night (when there is no light) and blocks Na⁺ during the day (when there is light). Creatine kinase catalyzes the major energy demanding reaction, whereas pyruvate kinase may regulate the pigmentation and phagocytosis. In addition, ATP synthase could be involved in mitochondrial dysfunction.

GFAP is a retinal stress indicator that regulates the breakdown of the blood-retinal barrier and the initial changes of Müller cells and the RPE. It was confirmed that oxidative stress, peroxiredoxin, and thioredoxin were increased in AMD by observing changes in chaperones, including heat shock proteins and albumin, and changes in endogenous antioxidants such as peroxiredoxin and thioredoxin.

The phosphorylation patterns of retinoid-binding proteins, such as CRABP, CRALBP, RDH, RGR, RPE65, and IRBP change. The expression of retinoid-binding proteins may induce perturbation of the circadian clock, which regulates cytoskeletal remodeling through actin, vimentin, and tubulin phosphorylation. Cytoskeletal modification regulates changes in the extracellular matrix regulated by MMP proteins and angiogenic responses mediated by VEGF and EPO.

During the progression of AMD, peripheral RPE showed an increase in apoptotic proteins. In particular, peripheral mitochondria were disrupted by prohibitin, which was depleted as a nuclear-mitochondrial shuttle in the RPE.

Activation of the inflammatory response by complement factor family members C3, C9, CFB, CFH, and CFI indicates that AMD involves an inflammatory response through vitronectin, clusterin, and plasminogen interactions.

### Experimental Example 5: Preparation of AMD-Related Protein Map

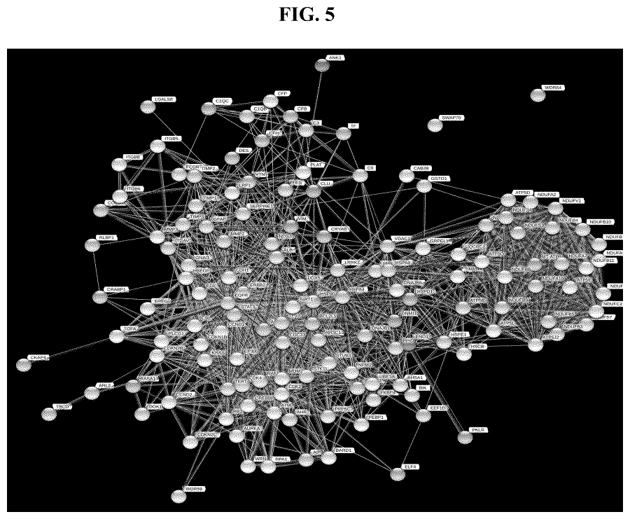

FIG. 5 is a protein map prepared based on the analysis of AMD-related proteins. A biomarker interaction map of AMD and oxidative stress was prepared using the following bioinformatics tools. Specifically, the biomarker interaction map was prepared using protein-protein interaction map software and databases, including STRING 10.0 (http://string-db.org/), MIPS (http://mips.helmholtz-muenchen.de/proj/ppi) and iHOP (http://www.ihop-net.org/UniPub/iHOP/). The STRING database currently deals with 9,643,763 proteins from 2,031 organisms. Proteins found under AMD or oxidative stress conditions were added to establish the AMD interactome. The AMD biomarker interactome was constructed using the 94 proteins discovered in this experiment.

Protein interactions were presented using eight categories, including neighborhood (green), gene fusion (red), co-occurrence (dark blue), co-expression (black), binding experiments (purple), databases (blue), homology (cyan), and text mining (lime). Protein interactions were determined and confirmed by genomic context, high-throughput experiments, co-expression, and previous publications of Pubmed. Protein database analysis showed the structure-specific phosphorylation of specific proteins in AMD eyes. The interaction between AMD proteomes was compared to the retina/RPE proteome under stress conditions. Lipids in the retina were extracted from retinal epithelial cell (ARPE-19) extracts using chloroform-methanol (2:1, v/v), an organic solvent was evaporated under a nitrogen stream, and the lipid was quantitatively analyzed by HPLC and mass spectrometry. Through this, the lipid changes in retinal cells were elucidated in an oxidative environment. The AMD biomarker map suggests that 68 to 81% of protein signals in the AMD mechanism may be initiated under oxidative stress.

The AMD map suggested cardiolipin as a mitochondrial indicator of cell apoptosis. The AMD map proposes that clumps of protein residues called drusens may include altered lipids including cholesterol.

Furthermore, through the AMD proteomic map, it was confirmed that the pathological mechanism of AMD includes one or more of the following (1) to (12):
(1) mitochondrial dysfunction in peripheral retinal cells (prohibitin, ATP synthase)
(2) oxidative stress caused by exposure to intense or prolonged light (peroxiredoxin, thioredoxin, glutathione S-transferase)
(3) cytoskeletal remodeling by microtubule, actin filament, and intermediate filament (vimentin, actin, tubulin)
(4) high concentration of nitric oxide (nitric oxide synthase)
(5) hypoxia (HIF1, erythropoietin, VEGF)
(6) circadian clock (melatonin) disruption
(7) apoptotic proteomes (pJAK2, pSTAT3, Bclxl, caspases)
(8) altered lipid concentrations (cardiolipin, cholesterol)
(9) visual cycle proteomes (RPE65, CRABP, CRALBP)
(10) altered energy metabolism (carnitine, pyruvic acid, ATP synthase)
(11) aggregation of crystallins
(12) increase in inflammatory proteomes (CFH, C3, collagen, vitronectin)

1. A composition for diagnosing age-related macular degeneration (AMD), comprising a preparation for measuring the mRNA or protein expression level of an age-related macular degeneration-related biomarker, wherein the age-related macular degeneration-related biomarker comprises:
a first biomarker comprising one or more selected from the group consisting of pyruvate kinase, α2 macroglobulin MG1 domain, PAK S/T kinase, protein phosphatase (PP2A), tyrosine protein kinase (FES/FPS), creatine kinase, vimentin, ubiquinone oxidoreductase, inositol 1,4,5 trisphosphate receptor, calponin, ankyrin repeat domain 30B, guanylate cyclase, complement component 3 (C3), complement component 9 (C9), complement factor H (CFH), vitronectin, retinal pigment epithelium-specific 65 (RPE65), retinal G protein coupled receptor (RGR), retinaldehyde binding protein (RLBP), mitochondrial protein involved in energy metabolism (ATPase), transcription factor (p53), protease (MMP), lamin, albumin, apoptotic protein (Annexin V), redox reaction (GST) protein, nuclear signaling factor (p53), nuclear signaling factor (BRCA1), nuclear signaling factor (ATM), oxygen-mediated apoptotic molecule (HIF), nuclear-mitochondrial signaling factor (VDAC), nuclear-mitochondrial signaling factor (EP300), ubiquitin, peroxiredoxin, MAP kinase, and Bub1/Bub3 complex; and/or
a second biomarker comprising one or more selected from the group consisting of transferrin, enolase, glial fibrillary acidic protein (GFAP), dynamin-like protein, WD protein 59, apoptotic molecule (Bcl2), apoptotic molecule (BAD), apoptotic molecule (BAX), apoptotic molecule (BAK), aging-related protein (prohibitin), cyclooxygenase (COX), nuclear signaling factor (SIRT1), oxygen-mediated apoptotic molecule (EPO), oxygen-mediated apoptotic molecule (Bcl), oxygen-mediated apoptotic molecule (BAD), oxygen-mediated apoptotic molecule (TXN), nuclear-mitochondrial signaling factor (PHB), and nuclear-mitochondrial signaling factor (RBP).

2. The composition of claim 1, wherein the preparation for measuring the mRNA expression level of the biomarker comprises one or more of a primer pair, a probe, and an antisense nucleotide, which specifically bind to the gene of the biomarker, and
the preparation for measuring the protein expression level of the biomarker comprises one or more of an antibody, an interacting protein, a ligand, nanoparticles, and an aptamer, which specifically bind to the protein or peptide fragment of the biomarker.

3. A kit for diagnosing age-related macular degeneration, comprising the composition of claim 1, wherein the kit is a reverse transcription polymerase chain reaction (RT-PCR) kit, a DNA chip kit, an enzyme linked immunosorbent assay (ELISA) kit, a protein chip kit, a rapid kit, or a multiple reaction monitoring (MRM) kit.

4. A method for providing information for diagnosing age-related macular degeneration, the method comprising:
(a) measuring the mRNA or protein expression level of an age-related macular degeneration-related biomarker in a biological sample of a subject;
(b) comparing the measured mRNA or protein expression level with that of a control sample; and
(c) diagnosing the subject as being likely to have age-related macular degeneration when the measured mRNA or protein expression level is increased or decreased compared to that of the control sample,
wherein the biomarker is one or more of the first and second biomarkers of claim 1.

5. The method of claim 4, wherein step (c) is a step of diagnosing the subject as being likely to have age-related macular degeneration when the mRNA or protein expression level of the first biomarker is increased compared to the control sample, or when the mRNA or protein expression level of the second biomarker is decreased compared to the control sample.

6. The method of claim 5, wherein the first biomarker comprises:
biomarker 1-1 comprising one or more selected from the group consisting of pyruvate kinase, α2 macroglobulin MG1 domain, PAK S/T kinase, protein phosphatase (PP2A), tyrosine protein kinase (FES/FPS), and creatine kinase; and
biomarker 1-2 comprising one or more selected from the group consisting of ubiquinone oxidoreductase, inositol 1,4,5 trisphosphate receptor, calponin, ankyrin repeat domain 30B, guanylate cyclase, complement component 3 (C3), complement component 9 (C9), complement factor H (CFH), vitronectin, retinal pigment epithelium-specific 65 (RPE65), retinal G protein coupled receptor (RGR), retinaldehyde binding protein (RLBP), mitochondrial protein involved in energy metabolism (ATPase), transcription factor (p53), protease (MMP), lamin, albumin, apoptotic protein (Annexin V), redox reaction (GST) protein, ubiquitin, peroxiredoxin, MAP kinase, and Bub1/Bub3 complex, and
the second biomarker comprises:
biomarker 2-1 comprising one or more selected from the group consisting of transferrin, enolase, glial fibrillary acidic protein (GFAP), dynamin-like protein, and WD protein 59; and
biomarker 2-2 comprising one or more selected from the group consisting of apoptotic molecule (Bcl2), apoptotic molecule (BAD), apoptotic molecule (BAX), apoptotic molecule (BAK), and cyclooxygenase (COX),
wherein when the biological sample is derived from the neuroretina of the subject, the mRNA or protein expression levels of biomarker 1-1 and/or biomarker 2-1 are compared, and
when the biological sample is derived from the retinal pigment epithelium (RPE) of the subject, the mRNA or protein expression levels of biomarker 1-2 and/or biomarker 2-2 are compared.

7. The method of claim 5, wherein the biomarker comprises one or more selected from the group consisting of a transferrin in which S678 and/or S409 are/is phosphorylated;
a pyruvate kinase in which S437 is phosphorylated;
WD protein 59 in which S375 and/or Y437 are/is phosphorylated; and
ankyrin repeat domain 30B in which S592 is phosphorylated.

8. A method for screening substances for treating age-related macular degeneration, the method comprising:
(a) treating neuroretinal cells or retinal pigment epithelial cells with a candidate substance;
(b) measuring the mRNA or protein expression levels of one or more of the first biomarker and second biomarker of claim 1 in the treated cells; and
(c) comparing the measured mRNA or protein expression levels with those of control cells to confirm whether the first biomarker is decreased and/or whether the second biomarker is increased.
